(19) United States
(12) Patent Application Publication
(10) Pub. No.: US 2012/0150147 A1
(43) Pub. Date: Jun. 14, 2012

(54) SYSTEM AND METHOD FOR TREATING ISCHEMIC STROKE

(75) Inventors: Aleksandr Leynov, Walnut Creek, CA (US); Dave Barry, Livermore, CA (US); Vikas Gupta, San Leandro, CA (US); Soe Ngwe, Fremont, CA (US); Arani Bose, New York, NY (US)

(73) Assignee: Penumbra, Inc., Alameda, CA (US)

(21) Appl. No.: 13/312,816

(22) Filed: Dec. 6, 2011

Related U.S. Application Data
(60) Provisional application No. 61/421,111, filed on Dec. 8, 2010.

(51) Int. Cl. A61M 25/10; B23P 17/04; A61F 2/01

(52) U.S. Cl. 604/509; 606/200; 29/428

(57) ABSTRACT

A system for restoring blood flow through an occluded vessel in the cerebrovasculature is disclosed. The system includes a tubular element constructed of shape memory material eccentrically mounted on the distal end of an introducer. The walls of the tubular element are defined by standards and connectors, and the standards are oriented at an angle to the longitudinal axis of the tubular element. A method of manufacture of the system includes cutting a tube of shape memory material, conferring a twist on the cut tube, and heat setting the tube. The method further includes affixing the shape set tube to an introducer. A method of treatment using the system is also described.

### CROSS-REFERENCES TO RELATED APPLICATIONS

This application claims the benefit of provisional application No. 61/421,111 (Attorney Docket No. 41507-713.101), filed on Dec. 8, 2010, the full disclosure of which is fully incorporated herein by reference.

### BACKGROUND OF THE INVENTION

1. Field of the Invention

The present invention relates generally to the field of medical treatment and, more particularly, to a system and method for treating ischemic stroke which involves restoring patency to a cerebral artery of a patient.

Stroke is a leading cause of death and disability and a growing problem to global healthcare. In the US alone, over 700,000 people per year suffer a major stroke and, of these, over 150,000 people die. Even more disturbing, this already troubling situation is expected to worsen as the “baby boomer” population reaches advanced age, particularly given the number of people suffering from poor diet, obesity and/or other contributing factors leading to stroke. Of those who a survive stroke, approximately 90% will suffer long term impairment of movement, sensation, memory or reasoning, ranging from mild to severe. The total cost to the US healthcare system is estimated to be over $50 billion per year.

Strokes may be caused by a rupture of a cerebral artery (“hemorrhagic stroke”) or a blockage in a cerebral artery due to a thromboembolism (“ischemic stroke”). A thromboembolism is a detached blood clot that travels through the bloodstream and lodges in a manner that obstructs or occludes a blood vessel. Between the two types of strokes, ischemic stroke comprises the larger problem, with over 600,000 people in the US suffering from with ischemic stroke per year. When such an obstruction occurs in a cerebral vessel, the result is a stroke and consequent cell death soon thereafter. The resulting symptoms of immobility and/or loss of function depend upon the location of the occlusion within the cerebrovasculature, and the severity of impact of ischemic stroke is directly related to the length of time blood flow is occluded in a particular cerebral vessel.

Ischemic stroke treatment may be accomplished via pharmacological elimination of the thromboembolism and/or mechanical elimination of the thromboembolism. Pharmacological elimination may be accomplished via the administration of thrombolytics (e.g., streptokinase, urokinase, tissue plasminogen activator (TPA)) and/or anticoagulant drugs (e.g., heparin, warfarin) designed to dissolve and prevent further growth of the thromboembolism. Pharmacologic treatment is non-invasive and generally effective in dissolving the thromboembolism. Notwithstanding these generally favorable aspects, significant drawbacks exist with the use of pharmacologic treatment. One such drawback is the relatively long amount of time required for the thrombolytics and/or anticoagulants to take effect and restore blood flow. Given the time-critical nature of treating ischemic stroke, any added time is potentially devastating. Another significant drawback is the heightened potential of bleeding or hemorrhaging elsewhere in the body due to the thrombolytics and/or anticoagulants.

Mechanical elimination of thromboembolic material for the treatment of ischemic stroke has been attempted using a variety of catheter-based transluminal interventional techniques. One such interventional technique involves combining mechanical disruption of the thromboembolism and removal of the thromboembolic material with an aspiration catheter. Other methods include attempts to mechanically remove the thrombus using a cork screw type device.

Regardless of the means of removal of a thromboembolism, a common urgency exists: to restore blood flow through the vessel as soon as possible after occlusion, in order to minimize cell death during the acute phase of stroke. The urgency remains during the initial treatment of a patient while physicians determine the desired course of treatment for permanent and complete elimination of the embolism.

For these reasons, it is an object of the invention herein to provide a means for temporarily restoring blood flow through a blocked cerebral vessel, prior to and/or during the procedures to more permanently and completely remove the blockage. It is a further object of the invention to remove embolic material from the vessel. It is a further object of the invention to provide a tubular device that can be readily tracked through the tortuous and fragile anatomy of the cerebrovasculature. It is a further object of the invention to provide a device that will load readily into a delivery catheter, will deploy readily within the cerebrovasculature at the site of an occlusion, and will be readily removable via the delivery catheter following restoration of sufficient blood flow. It is a further object of the invention to permit the delivery and deployment of additional therapies (such as, for example, disruption and aspiration of the embolism) during use of the tubular device. At least some of these objections will be met by different aspects of the present invention as described below.

2. Description of the Background Art

U.S. Pat. No. 7,931,659 describes a thromboembolic removal system comprising a tubular receiver on the distal end of an elongate introducer. The receiver is intended to envelope and remove clot and occlusions in the cerebral vasculature. U.S. Patent Publication 2007/0239261 describes an aneurysm occlusion device which is positionable across an open neck of a cerebral aneurysm. The occlusion device optionally includes helical standards as shown, for example, in FIG. 3E.

### SUMMARY OF THE INVENTION

The present invention provides methods and apparatus for restoring patency in cerebral arteries blocked with thrombus, particularly in patients presenting with symptoms of occlusive stroke. The methods and apparatus herein allow for rapid deployment and restoration of blood flow in order to reduce the risk of permanent impairment and disability in patients suffering from occlusive stroke. By rapidly opening a region of occlusive thrombus within the patient's cerebral vasculature and restoring blood flow, the thrombus may resolve itself without additional treatment and/or there's an opportunity to provide alternative therapies, such as the delivery of thrombolytics in order to dissolve the clot while blood flow is maintained using the methods and systems herein. The present invention is particularly advantageous since it allows access to and deployment within thrombus which is occluding even highly tortuous regions of the cerebral arteries which are difficult to access with other treatment tools.

In a first aspect of the present invention, methods for restoring patency in a cerebral blood vessel occluded by thrombus comprise advance a radially constrained tubular element through the thrombus using an elongate pusher. The tubular element is released from constraint within the thrombus in order to open the thrombus and to provide a blood flow passage therethrough. A proximal end of the tubular element remains attached to and constrained by the elongate pusher even after a distal portion has been deployed. At least a portion of the proximal end of the tubular element near the elongate pusher is open or perforate so that blood may flow through the tubular element while the element remains expanded and attached to the pusher. In most instances, the entire tubular element is formed from an open or perforate scaffold or matrix which allows blood flow therethrough. After the thrombus has been resolved, either through use of the tubular element alone or optionally with additional thrombolytic or other treatments, the tubular element may be re-constrained or otherwise recaptured and withdrawn from the thrombus using the elongate pusher.

In preferred embodiments of the methods herein, the tubular element is deployed within and conforms to a curved portion of the cerebral blood vessel being treated. The curved portion may be highly curved, for example having a radius of curvature (measured on a center line of the blood vessel) less than 10 mm, often less than 7 mm, and sometimes below 5 mm. Particularly suitable structures for the tubular element include a plurality of helical standards or struts extending in a generally proximal to distal axial direction (where the axis is defined by the attached pusher element) and further includes a multiplicity of expandable connectors extending laterally between adjacent helical standards. In preferred embodiments, the expandable connectors comprise V-shaped connectors which open as the tubular element expands and which close as the tubular element is radially constrained.

In a second aspect, the present invention provides apparatus for restoring patency in a cerebral blood vessel. The apparatus comprises an elongate pusher having a proximal end and a distal end, where the elongate pusher is adapted for intravascular advancement into the cerebral vasculature. A self-expanding tubular element having a proximal end, a distal end, and a lumen therethrough, is attached at its proximal end to the distal end of the elongate pusher. The tubular element is self-expanding (that is, it may be radially constrained and will expand under its own resiliency to its fully expanded configuration when the constraint is relieved) from a constrained configuration to an expanded configuration. The proximal end of the tubular element is fixedly attached to the distal end of the elongate pusher since the tubular element is not intended to be permanently deployed or implanted within the vasculature. The tubular element typically comprises a plurality of helical standards and a multiplicity of expandable connectors extending between adjacent helical standards. The preferred configuration for the expandable connectors was described above in connection with the methods of the present invention. The apparatus further comprises a restraining sheath slidably disposed over the tubular element, where the, sheath may be advanced to radially restrain or constrain the tubular element and to be retracted in or to release the tubular element and allow the tubular element to self-expand to its fully deployed configuration.

In specific embodiments, the tubular element includes from two to eight helical standards or struts, typically from two to six, usually from two to four and most often having three helical standards or struts. The connectors and helical standards or struts have a width usually between about 0.0012 inch and 0.0018 inch and are typically composed of a shape memory or heat memory material, such as a nickel-titanium alloy. The helical standards will typically be oriented at an angle relative to the longitudinal axis which is usually in the range from 25° to 45°.

In a third aspect of the present invention, the apparatus for restoring patency as described above, may be manufactured from a tube composed of a desired shape memory material, such as a nickel-titanium alloy. The tube is cut according to a predetermined pattern to define generally axial standards and laterally expandable connectors extending between adjacent standards. The tube is then twisted about its longitudinal axis in order to impart a helical twist to the standards, and the tube is then heat set to retain the desired helical twist. After forming, the tubular element is connected to a rod-like introducer or pusher element. Thus, when released from constraint, the tubular member will self-expand along most of its distal and middle length. The proximal end near the attachment point to the elongate pusher will remain constrained by virtue of its attachment to the pusher. The assembly of the tubular element and elongate pusher are then combined with a radially constraining sheath which is placed over the tubular member to constrain the tubular member so that the apparatus may be delivered to a target site within the cerebral vasculature as described above.

### BRIEF DESCRIPTION OF THE DRAWINGS

Many advantages of the present invention will be apparent to those skilled in the art with a reading of this specification in conjunction with the attached drawings, wherein like reference numerals are applied to like elements and wherein:

FIG. 1 is a side elevation view of an embodiment of the invention in a deployed configuration within a straight vessel.

FIG. 2 is a side elevation view of an embodiment of the invention in a partially deployed configuration within a straight vessel.

FIG. 3 is a side elevation view of an embodiment of the invention deployed within thromboembolic material in a straight vessel.

FIG. 4A illustrates a cross-sectional end view of the embodiment of FIG. 3 within a model of a straight vessel, before deployment.

FIG. 4B illustrates a cross-sectional end view of the embodiment of FIG. 3 within a model of a straight vessel, after deployment.

FIG. 5 is a side elevation view of an embodiment of the invention deployed within a curved vessel.

FIG. 6 is a plan view of an “unrolled” embodiment according to the invention, illustrating an “as cut” pattern, before the device is shape set into a helical form.

FIG. 7 is a plan view similar to FIG. 6 showing the device after it has been shape set to include a right hand twist.

FIG. 8 is a plan view similar to FIG. 6 showing the device after it has been shape set to include a left hand twist.

FIG. 9 is a plan view of an alternative “unrolled” embodiment according to the invention, illustrating an “as cut” pattern before the device has been shape set into a helical form.

FIG. 10 is a perspective view of the embodiment of FIG. 9 in its deployed configuration after it has been shape set but before it has been mounted on a pusher tube.

### DETAILED DESCRIPTION OF THE INVENTION

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be routine undertaking for those of ordinary skill in the art having the benefit of this disclosure. The thromboembolic removal system disclosed herein boasts a variety of inventive features and components that warrant patent protection, both individually and in combination.

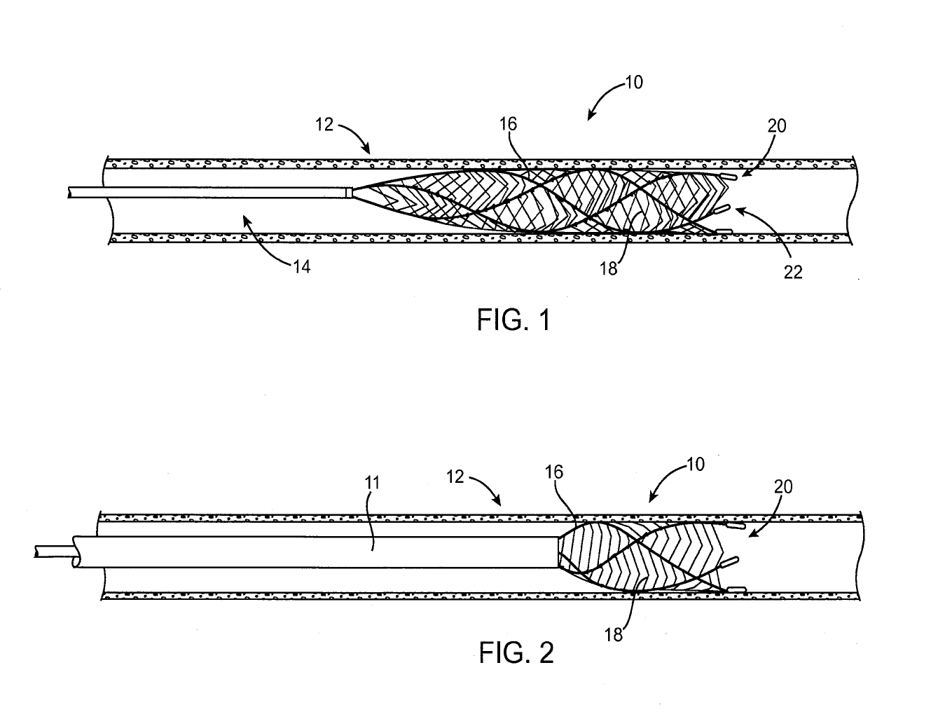

FIG. 1 illustrates an exemplary embodiment of a device according to the invention. Tubular intravascular device 10 is illustrated in its deployed configuration in a vessel 12. Its primary components are tubular element 20 mounted upon the distal end of pusher 14. In this example, tubular element 20 is affixed to pusher 14, so that tubular element 20 can be both introduced and withdrawn via pusher 14. Consequently, tubular element 20 can be readily repositioned within the vasculature. Moreover, tubular element 20 can be utilized in a temporary fashion during treatment of the acute phase of ischemic stroke. The device 10 is proportioned to be utilized within the cerebral vasculature including, but not limited to, the Internal Carotid Artery, External Carotid Artery, Vertebral Artery, Basilar Artery, Middle Cerebral Artery, Anterior Cerebral Artery, and the Posterior Cerebral Artery. Preferred devices are expandable to an outer diameter in the range of between 2 mm and 6 mm according to vessel size.

The device may be constructed from any number of compositions having suitable biocompatibility and strength characteristics, and may be dimensioned in any number of suitable sizes and lengths depending upon the location of the thromboembolism, variances in patient anatomy, and the size and shape of the thromboembolism. Device 10 of FIG. 1 may be used alone or in conjunction with other therapies and devices for disruption and removal of a thromboembolism. Accordingly, tubular element 20 and pusher 14 are proportioned to extend through the lumen of a delivery and aspiration catheter (not pictured). Tubular element 20 may advantageously be mounted eccentrically to the pusher or delivery wire in order to permit the contemporaneous operation of additional therapeutic devices. Although a pusher catheter having black Pebax HS tubing is illustrated in FIG. 1, other materials and alternative dimensions may be suitable according to the invention. The pusher 14 may alternatively be a non-tubular delivery wire, depending upon the requirements needed to deliver the tubular element 20 to the treatment site (not shown). Tubular element 20 includes a plurality of helical standards or struts 16, and a multiplicity of V-shaped connectors 18 span the regions between adjacent standards and define the “walls” and central lumen 22 of the tubular element. These and other specific features of tubular element 20 will be discussed in greater detail below with reference to FIGS. 6-10. In the embodiment illustrated in FIG. 1, tubular element 20 is constructed from a nickel-titanium alloy, such as Nitinol® with “shape memory” or “superelastic” characteristics. The tubular element 20 is consequently capable of being retained in a constrained form or shape prior to deployment.

FIG. 2 illustrates partially deployed tubular element 20 on a device 10 that has previously been loaded into a delivery sheath 11. Tubular element 20 is crimped into a low profile delivery configuration and maintained in the sheath 11 for advancement into the vasculature. Delivery sheath 11 retains tubular element 20 in a low profile (radially constrained or compressed) configuration during tracking of the device 10 under fluoroscopic visualization to a treatment site within the vasculature of a subject. Sheath 11 is an elongate tubular catheter preferably formed of a polymeric material such as pebax, nylon, urethane, PTFE, polyimide, metals such as stainless steel, platinum etc., or other suitable materials. A central lumen extends throughout the length of the sheath 11. The sheath 11 is proportioned for passage through the cerebral vasculature and may have an outer diameter in the range of 0.032 inch to 0.065 in. When device 10 is properly positioned within the vasculature of a subject, sheath 11 is withdrawn (and is shown partially withdrawn in FIG. 2), permitting tubular element 20 to self-expand and deploy to its unconstrained configuration to engage the vascular walls.

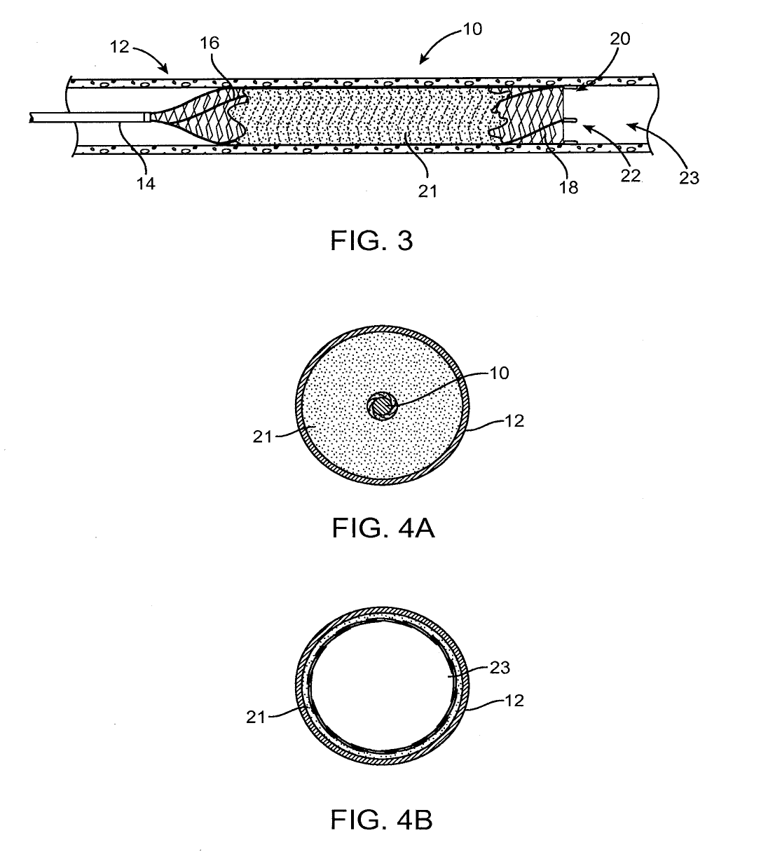

This unconstrained configuration is shown in FIG. 3, which illustrates device 10 fully deployed within a vessel model 12. Prior to introduction of device 10, thrombus 21 was placed into lumen 23 of a blood vessel 12. According to the procedure described above in relation to FIG. 2, device 10 in its delivery configuration is advanced through vessel lumen 23 and through a length of thrombus 21. The delivery sheath 11 is withdrawn (as shown in FIG. 2), and the tubular element 20 expands radially to the deployed configuration shown in FIG. 3. The “walls” of tubular element 20 now define a lumen 22 and engage or line the inner walls of the vessel lumen 23. Patency of the vessel lumen 23 is thereby restored through thrombus 21.

The restoration of patency of vessel lumen 23 can be seen in FIGS. 4A and 4B. An end view of device 10 is illustrated before and after deployment within a straight vessel model. In FIG. 4A, vessel lumen 23 is occluded or blocked by thrombus 21. Device 10 is positioned within a central portion of thrombus 21, as illustrated in FIG. 4A, and preferably extends through the length of thrombus 21. Tubular element 20, upon deployment by retraction of sheath 11 (FIG. 2), expands radially within a central portion of thrombus 21, increasing the diameter of a bore through the thromboembolic material of thrombus 21, as the “walls” of tubular element 20 approach the inner walls of vessel lumen 23. As a result of deployment, tubular element 20 defines the device lumen within thrombus 21, thereby restoring patency to vessel lumen 23. The distal opening of device lumen 22 can be seen in FIG. 4B. When device 10 is utilized in the lumen of a subject, contrast dye may be injected at any time during the procedure in order to ascertain whether patency has been restored to the lumen. Device 10 can subsequently be retracted back into a sheath (not pictured) when the treating physician elects to withdraw the device from the lumen of the subject.

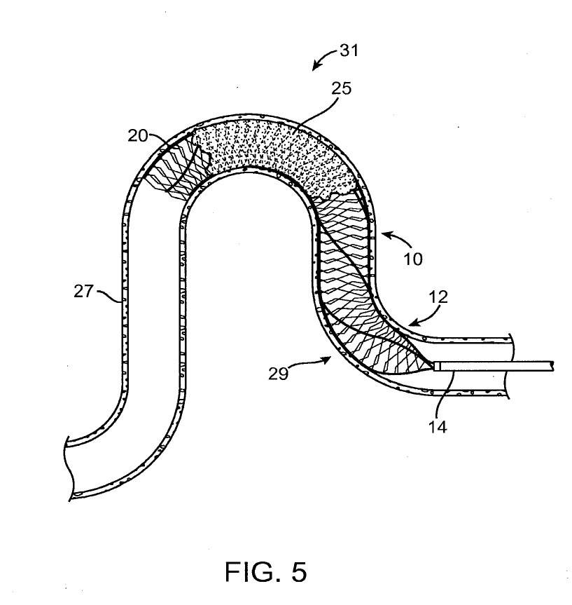

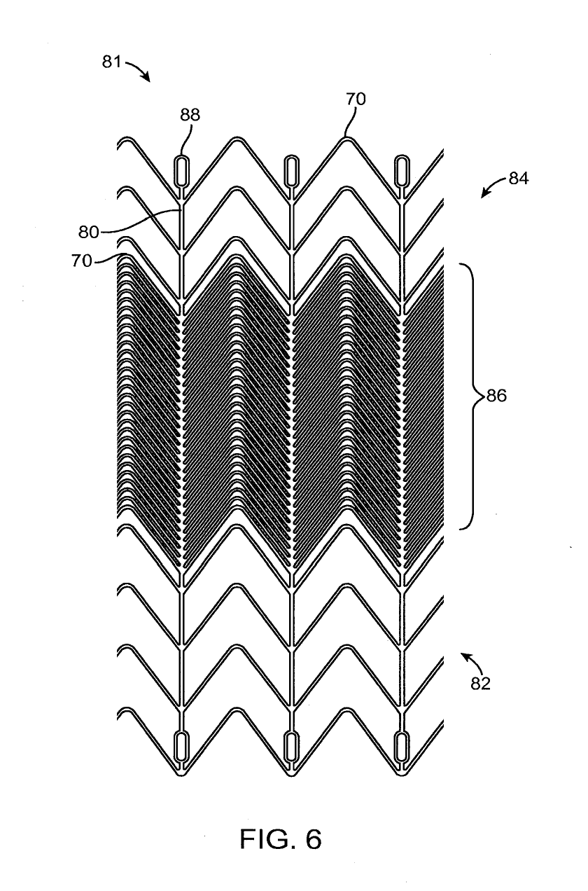

Among the advantages of the invention herein are its superior, kink-resistant, reversible trackability and reversible deployability within tortuous vasculature. In order to illustrate the superior tracking and reliable deployment of the system, device 10 is shown deployed within a curved vessel 27 in FIG. 5. Thrombus 25 in the vessel 27 is disposed distally to a first curved portion 29 and into a second curved portion 31. Device 10 is tracked across both the first curved portion 29 and the second curved portion 31, and tubular element 20 is deployed within a central portion of the curved thrombus 25. Following placement of tubular element 20 within and extending through the length of the thrombus 25, the delivery sheath, (not pictured) may be withdrawn. After withdrawal of the delivery sheath, tubular element 20 is permitted to expand radially outwardly to closely meet the inner surfaces of the walls of the vessel 27. Despite the curved configuration of the vessel 27, tubular element 20 readily deploys to restore patency to the lumen. Furthermore, device 10 can be readily withdrawn back into the delivery sheath in order to reposition device 10 or to remove it completely from the vessel. As can be seen in FIG. 5, the helical configuration of the standards 16 and lateral expandability of the V-shaped connectors 18 provide an enhanced conformability for the tubular element 20 within even highly curved vasculature.

The specific features of tubular element 20 facilitate tracking, positioning, repositioning, deployment and removal within and throughout curvatures such as those illustrated in FIG. 5. Tubular element 20 may be formed by laser cutting features into a length of Nitinol tubing, and then conferring a twist and shape-setting the material one or more times using methods known to those skilled in the art. For example, a tube of 3.5 mm outer diameter and 0.005 inch thickness may be cut in a predetermined pattern. Examples of suitable patterns are illustrated in FIGS. 6-10.

FIG. 6 represents tubular element 81 as though the patterned tube were cut along its length and then laid flat. In this configuration, the device design consists of three uprights, standards, or elongate members 80. As shown in FIG. 6, elongate members 80 extend from the proximal portion 82 to the distal portion 84. The elongate members 80 provide axial strength to the central portion 86. The elongate members may also be used to provide axial force to the device if it is necessary to re-position the device after a partial deployment within the vessel as discussed above. Each elongate member may include one or more eyelets 88. V-shaped connectors 70 extend between elongate members 80.

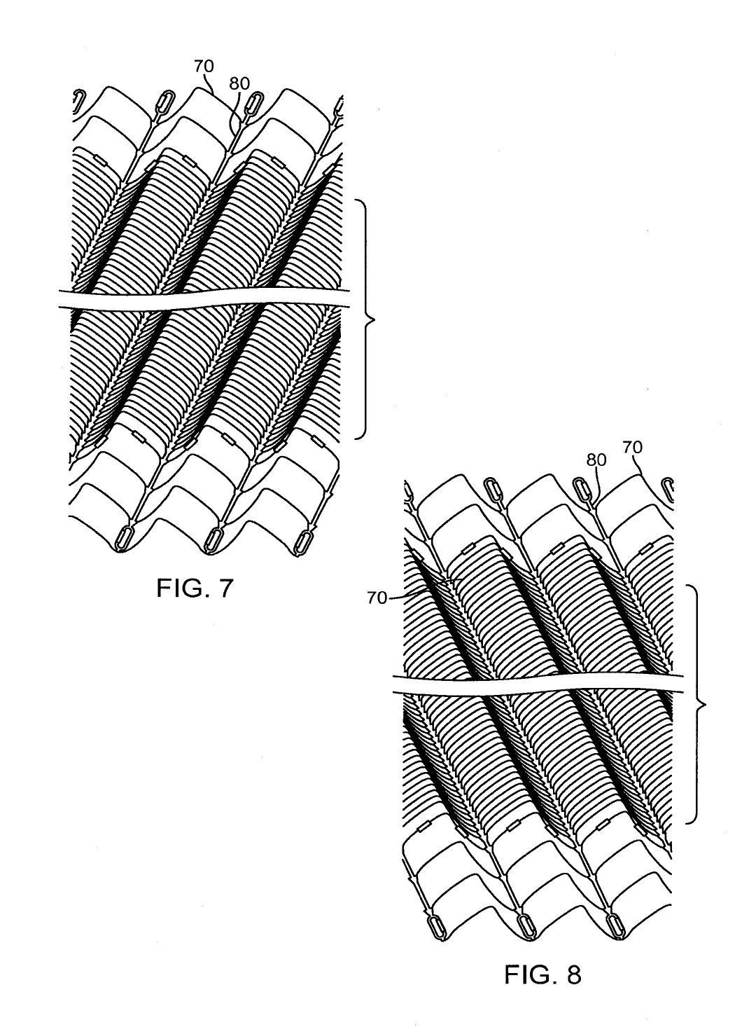

After the design is laser cut into a nickel-titanium tube, the tube is then twisted and shape set to helically position the uprights, or elongate members 80. It has been found that a helical arrangement helps the deployed device conform to the vessel walls, and it also improves the ability of the device to resist kinking. FIG. 7 illustrates the device as it would appear longitudinally cut and laid flat following shape setting using a right hand twist. FIG. 8 is a similar drawing of the device as it would appear following shape setting using a left hand twist.

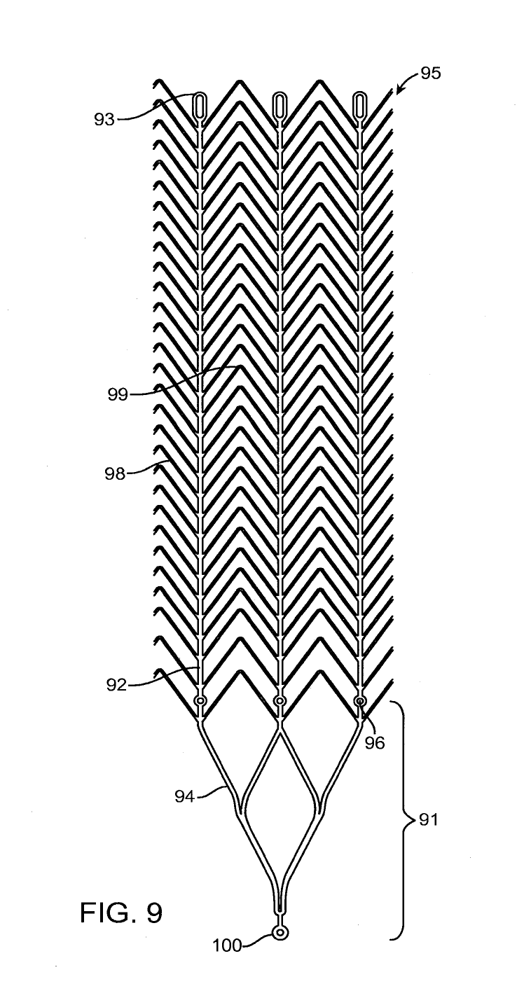

An example of another suitable pattern is illustrated in FIG. 9. In FIG. 9, patterned tubular element 95 is shown as though the tube were cut along a longitudinal axis and laid flat. In the alternative embodiment of FIG. 9, three standards 92 may be used. Coupled between standards 92 are generally V-shaped strut members 98, with apexes 99 extending towards the distal end 95. Struts that form V-connectors 98 have widths that may vary between 0.001 inch and 0.00175 inch. Connectors 98 may have a broadened region and one or more tapered regions. Connectors 98 help to maintain the cylindrical shape of the tubular element 95, and also facilitate collapsing of the device for loading of the device into a sheath by providing folding points for the device. To fold the device for insertion into the sheath, tension is applied in a proximal direction, causing the device to fold along the apexes of the strut members 98 and to thus place the device in a radially compressed configuration. Each standard 92 includes eyelet 93 at distal end 95. Legs 94 extend from the proximal ends of a plurality of the standards 92. Each of the legs 94 includes an eyelet 96. Tubular element 95 may also include mounting terminus 100.

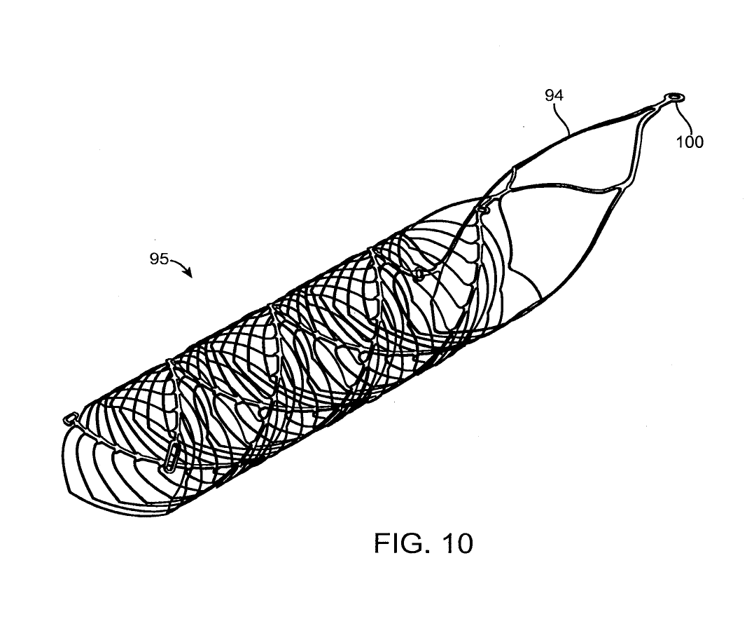

After a pattern such as that illustrated in FIG. 9 is cut into the nickel-titanium tube, a circumferential twist is applied to the tube. Such a twist will confer a twist on standards 92 at a predetermined angle of between 31 and 47 degrees to the circumference of the tube. A range of twist angles of between 33° and 45° was tested (see Table 1.) Examples of specific embodiments tested are set forth in greater detail below. Generally, the application of a circumferential twist during the manufacture of a device according to the invention confers advantages in tracking and deployment of the device, especially within tortuous anatomy, such as that illustrated in a glass model in FIG. 5. Delivery and deployment of a device according to the invention within a bend or curve of a vessel is uniform and kink-resistant. The twist will be heat set into the “memory” of the material, and tubular element 95 will appear as illustrated in FIG. 10. Following twist and shape set, tubular element 95 will be mounted to the distal end of a pusher (not pictured) via mounting terminus 100 to form a complete device. The length of tubular element 95, shown in its deployed configuration in FIG. 10, may vary between 9.0 mm and 30 mm, but preferably is longer than the length of the thromboembolism.

Prior to delivery and deployment of the completed device, the tubular element will be collapsed, crimped down or otherwise reduced to its delivery configuration and restrained therein as described above. In preparation for treating a subject, the device within its sheath will be loaded in a delivery catheter. During a procedure performed under fluoroscopic visualization, the delivery catheter is tracked to the site of the occlusion. The distal end of the catheter is tracked through the occlusion until the distal tip thereof extends beyond the occlusion.

The sheath is then withdrawn as described above to allow partial or complete expansion of the device within the vessel. Additional therapeutics, such as pharmacologic agents, may be administered before and/or during deployment if desired by the physician. In addition, or alternatively, additional mechanical means for removal of thromboembolic material may be deployed while the device is in place within the lumen. Further, expansion of the device may be increased incrementally during use. Contrast dye may be injected at any point during deployment of the device to determine the extent of restoration of blood flow. When blood flow is restored to the satisfaction of the physician, the device may be resheathed and removed from the vessel. Additional treatment, whether pharmacologic or mechanical, may continue or commence according to the treating practitioners' determination.

### Example 1

A nitinol tube was cut according to the pattern illustrated in FIG. 9. Resulting strut widths were in a range of between 0.0014-0.0015 inch. The cut tube was mounted to a pusher catheter with 0334, black Pebax HS tubing. A circumferential twist was applied to the cut tube, resulting in a twist angle of approximately 33°. The device was collapsed to a delivery configuration and sheathed, and loaded into a Penumbra 041 delivery catheter. A straight glass tube was filled with blood clot derived from food source animal product. The delivery catheter was positioned through the clot within the glass model, and the sheath withdrawn to deploy the scaffold within the clot. Patency was restored in the model lumen as a result.

### Example 2

A nitinol tube was cut according to the pattern illustrated in FIG. 9. Resulting strut widths were in a range of between 0.00135-0.0015 inch. The cut tube was mounted to a pusher catheter with 0334, black Pebax HS tubing. A circumferential twist was applied to the cut tube, resulting in a twist angle of approximately 30°. The device was collapsed to a delivery configuration and sheathed, and loaded into a Penumbra 032 delivery catheter. A blood clot derived from food source animal product was obtained and treated with hot water in order to harden the clot. The hardened clot was placed within a circuitous glass tube, and the clot was placed within a curve in the glass model. The delivery catheter was positioned through the bend and through clot within the model, and the sheath withdrawn to deploy the scaffold within the clot. Patency was restored in the model lumen as a result.

Further examples are illustrated in Table 1 below.

TABLE 1

| Prototype | | Twist Angle | |
|---|---|---|---|
| # | Strut Width (in.) | (°) | OD (mm) |
| 1 | .0014-.0015 | 33 | 5.1 |
| 2 | .00135-.0015 | 30 | 4.81 |
| 3 | .0012 | 34 | 4.92 |
| 4 | .00135-.0015 | 31 | 4.82 |
| 5 | .00135-.0015 | 36 | 5.1 |
| 6 | .0015 | 40 | 5.0-5.2 |
| 7a | .00165-.00175 | 44-45 | 5.0-5.2 |
| 7b | .0015-.0016 | 44 | 5.0-5.2 |

While the invention may be modified and alternative forms may be used, specific embodiments of the invention have been illustrated and described in detail. It should be understood, however, that the description herein of specific embodiments is not intended to limit the invention to the particular forms disclosed. The invention and following claims are intended to cover all modifications and equivalents falling within the spirit and scope of the invention.

What is claimed is:

1. A method for restoring patency in a cerebral blood vessel occluded by thrombus, said method comprising:
advancing a radially constrained tubular element through the thrombus using an elongate pusher;
releasing the tubular element from constraint within the thrombus, wherein a proximal end of the tubular element remains attached to and constrained by the elongate pusher and wherein at least a proximal portion of the tubular element near the elongate pusher allows blood flow through the tubular element while the element remains expanded and attached to the elongate pusher;
re-constraining the tubular element; and
withdrawing the re-constrained tubular element from the thrombus using the elongate pusher.

2. A method as in claim 1, wherein the tubular element is deployed within and conforms to a curved portion of the cerebral blood vessel.

3. A method as in claim 2, wherein the curved portion of the cerebral vasculature has a radius of curvature less than 10 mm.

4. A method as in claim 1, wherein the tubular element comprises a plurality of helical standards extending generally in a proximal to distal direction and a multiplicity of expandable connectors extending between said helical standards.

5. A method as in claim 4, wherein the expandable connectors extending between the adjacent helical standards comprise V-shaped connectors which open as the tubular element expands and which close as the tubular element is radially constrained.

6. A method as in claim 1, further comprising delivering a thrombolytic agent while the tubular element remains expanded within the thrombus.

7. Apparatus for restoring patency in a cerebral blood vessel, said apparatus comprising:
an elongate pusher having a proximal end and a distal end, wherein said elongate pusher is adapted for intravascular advancement to the cerebral vasculature;
a self-expanding tubular element having a proximal end, a distal end, and a lumen therethrough, wherein the tubular element is self-expanding from a constrained configuration to an expanded configuration, wherein the proximal end of the tubular element is fixedly attached to the distal end of the elongate pusher, and wherein the tubular element comprises a plurality of helical standards and a multiplicity of expandable connectors extending between adjacent helical standards; and
a restraining sheath slidably disposed over the tubular element, wherein the sheath radially restrains the tubular element when fully advanced and releases the tubular element when fully retracted except for the proximal end of the tubular element which remains constrained by attachment to the distal end of the elongate pusher.

8. Apparatus as in claim 7, wherein the tubular element includes from 2 to 8 helical standards.

9. Apparatus as in claim 8, wherein the expandable connectors extending between the adjacent helical standards comprise V-shaped connectors which open as the tubular element expands and which close as the tubular element is radially constrained.

10. Apparatus as in claim 9, wherein said connectors and elongate elements have a width between 0.0012 inch and 0.0018 inch.

11. Apparatus as in claim 7, wherein said tubular element is comprised of a shape memory material, said tubular element is shape set in its expanded deployed confiruation.

12. Apparatus as in claim 7, wherein said helical standards are oriented at an angle to said longitudinal axis when said tubular element is in said deployed configuration.

13. Apparatus as in claim 12, wherein said angle is between 25° and 45°.

14. A method of manufacture of a system for restoring patency to a cerebral vessel, the method comprising the steps of:
providing a tube constructed of shape memory material;
cutting the tube according to a predetermined pattern to define standards and connectors;
applying a circumferential twist to the cut tube;
heat setting said cut tube having a circumferential twist; and
attaching the heat set cut tube to an elongate pusher.

15. The method according to claim 14, wherein the method further comprises the additional steps of:
affixing the tube to an introducer.

16. The method according to claim 15, wherein said step of affixing the tube to an introducer comprises affixing the tube in an eccentric fashion.

17. The method according to claim 14, wherein the standards or connectors comprise a width of between 0.0012 inch and 0.0018 inch.

18. The method according to claim 14, wherein said twist is in the range of between 25° and 45°.
